(19) United States
(12) Patent Application Publication
(10) Pub. No.: US 2010/0063479 A1
(43) Pub. Date: Mar. 11, 2010

(54) SMALL PROFILE, TUBULAR COMPONENT DESIGN AND METHOD OF MANUFACTURE

(75) Inventors: KENNETH MERDAN, Loretto, MN (US); BANI MOHANTY, Woodbury, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/207,800

(22) Filed: Sep. 10, 2008

(51) Int. Cl. A61M 25/09; C21D 1/40; B23P 17/00

(52) U.S. Cl. 604/528; 219/162; 29/527.1

(57) ABSTRACT

Medical devices and methods for making and using the same. An example method for manufacturing a medical device may include providing a metallic tubular member, cutting a plurality of slots in the tubular member, and chemically treating at least a portion of the tubular member to remove material from the tubular member and define a treated portion. The tubular member may include an outer diameter and an inner diameter. The slots may have a slot width. The treated portion may have one or more of a decreased outer diameter, an increased inner diameter, or an increased slot width in at least some of the slots.

### FIELD OF THE INVENTION

The present invention pertains to medical devices, and methods for manufacturing medical devices. More particularly, the present invention pertains to elongated intracorporeal medical devices including a slotted tubular member.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example method for manufacturing a medical device may include providing a metallic tubular member, cutting a plurality of slots in the tubular member, and chemically treating at least a portion of the tubular member to remove material from the tubular member and define a treated portion. The tubular member may include an outer diameter and an inner diameter. The slots may have a slot width. The treated portion may have one or more of a decreased outer diameter, an increased inner diameter, or an increased slot width in at least some of the slots.

Another example method for manufacturing a medical device may include providing a nickel-titanium alloy tubular member, laser cutting a plurality of slots in the tubular member, and chemically etching at least a portion of the tubular member to remove material from the tubular member and define a treated portion. The tubular member may have an outer diameter and an inner diameter. The slots may have a slot width. The treated portion may have a decreased outer diameter, an increased inner diameter, and an increased slot width in at least some of the slots.

An example medical device may include an elongate shaft including a metallic tubular member. The tubular member may have a length, an inner diameter, an outer diameter, and a plurality of slots formed therein. The inner diameter may vary along the length of the tubular member. The outer diameter may also vary along the length of the tubular member.

Another example method for manufacturing a medical device may include providing a tubular member, cutting a plurality of slots in the tubular member, and chemically treating at least a portion of the tubular member to remove material from the tubular member and define a treated portion. The tubular member may include an outer diameter and an inner diameter. The slots may have a slot width. The treated portion may have one or more of a decreased outer diameter, an increased inner diameter, or an increased slot width in at least some of the slots.

Another example medical device may include an elongate shaft including a tubular member. The tubular member may have a length, an inner diameter, an outer diameter, and a plurality of slots formed therein. The inner diameter may vary along the length of the tubular member. The outer diameter may also vary along the length of the tubular member.

Another example method for manufacturing a medical device may include providing a tubular member, forming a plurality of pockets in the tubular member, and chemically treating at least a portion of the tubular member to remove material from the tubular member and define a treated portion. The tubular member may include an outer diameter and an inner diameter. The pockets may have a pocket width. The treated portion may have one or more of a decreased outer diameter, an increased inner diameter, or an increased pocket width in at least some of the pockets.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example medical device disposed in a blood vessel;

FIG. 2 is a partial cross-sectional view of an example medical device;

FIG. 2A is a cross-sectional side view of an example tubular member for use in a medical device;

FIG. 3A is a side view of another example tubular member for use in a medical device prior to chemical treatment;

FIG. 3B is a side view of the tubular member illustrated in FIG. 3A after chemical treatment;

FIG. 3C is a side view of another example tubular member;

FIG. 4 is a plan view of an example bathing apparatus;

FIG. 4A is a plan view of another example bathing apparatus; and

FIG. 5 is a partial cross-sectional view of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

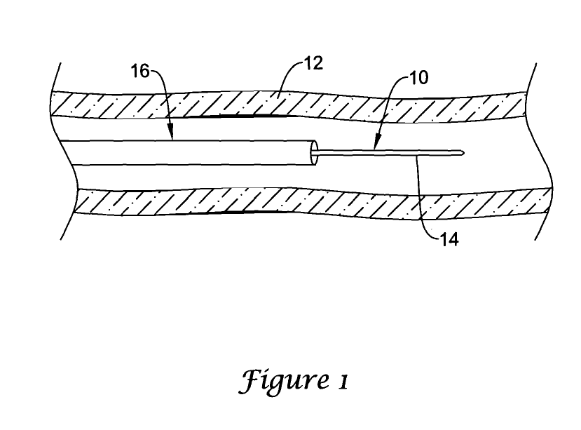

FIG. 1 is a plan view of an example medical device 10, for example a guidewire, disposed in a blood vessel 12. Guidewire 10 may include a distal section 14 that may be generally configured for use within the anatomy of a patient. Guidewire 10 may be used for intravascular procedures. For example, guidewire 10 may be used in conjunction with another medical device 16, which may take the form of a catheter, to treat and/or diagnose a medical condition. Of course, numerous other uses are known amongst clinicians for guidewires, catheters, and other similarly configured medical devices.

Although medical device 10 is depicted in several of the drawings as a guidewire, it is not intended to be limited to just being a guidewire. Indeed, medical device 10 may take the form of any suitable guiding, diagnosing, or treating device (including catheters, endoscopic instruments and/or endoscopes, laparoscopic instruments, stent delivery systems, embolic filter systems, urology stone retrieval systems, embolic coil delivery systems, atherectomy shafts, thermoctomy shafts, pacing leads, neuromodulation contacts, neuromodulation electrodes, cardiac rhythm management leads and/or contacts, etc., and the like) and it may be suitable for use at essentially any location and/or body lumen within a patient. For example, medical device/guidewire 10 may be suitable for use in neurological interventions, coronary interventions, peripheral interventions, etc. As such, guidewire 10 may be appropriately sized for any given intervention. For example, guidewire 10 may have an outside diameter of about 0.001 to 0.5 inches or about 0.0015 to 0.05 inches (e.g., about 0.010 to 0.014 inches) for neurological interventions; an outside diameter of about 0.001 to 0.5 inches or about 0.01 to 0.05 inches (e.g., about 0.014 inches) for coronary interventions; or an outside diameter of about 0.01 to 0.5 inches or about 0.02 to 0.05 inches (e.g., about 0.014 to 0.038 inches) for peripheral interventions. These dimensions, of course, may vary depending on, for example, the type of device (e.g., catheter, guidewire, etc.), the anatomy of the patient, and/or the goal of the intervention. In at least some embodiments, for example, guidewire 10 may be a crossing guidewire that can be used to help a clinician cross an occlusion or stenosis in vessel 12.

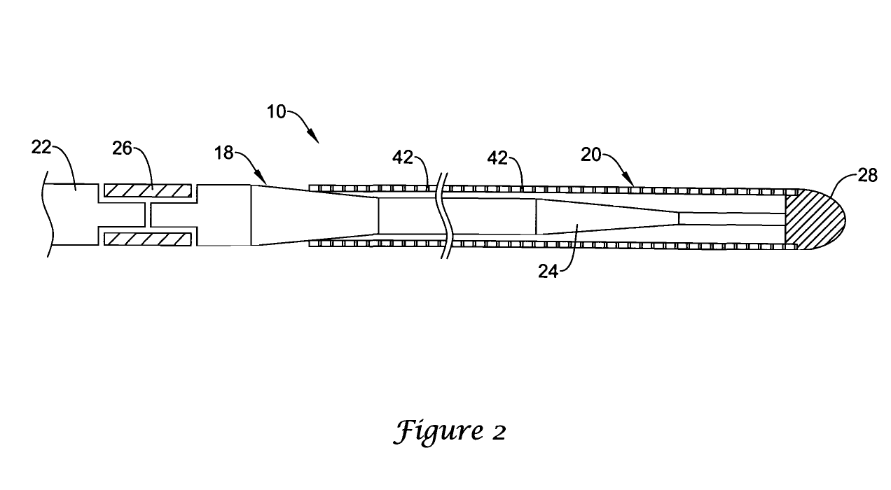

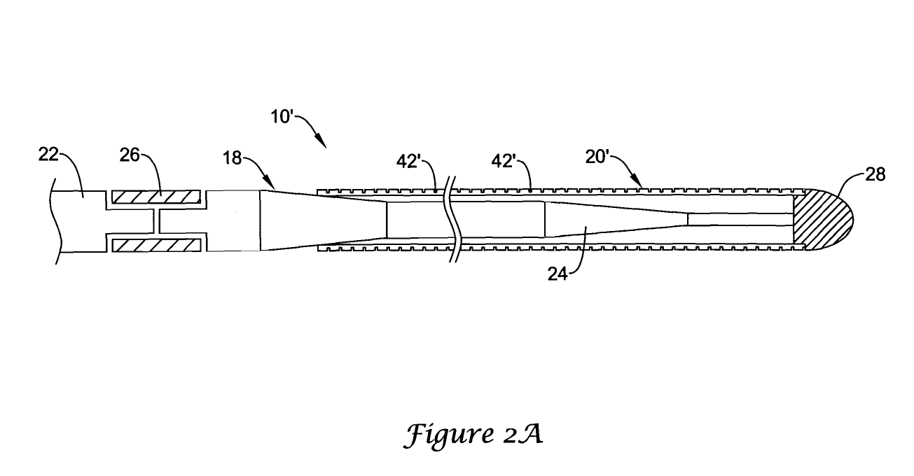

FIG. 2 is a partial cross-sectional view of guidewire 10. Here it can be seen that guidewire 10 may include a core member or core wire 18 and a tubular member 20 disposed over at least a portion of core wire 18. Core wire 18 may include a proximal section 22 and a distal section 24. A connector 26 may couple or otherwise attach proximal section 22 to distal section 24. Alternatively, core wire 18 may be a unitary member without a connector. A tip member 28 may also be coupled to core wire 18 and/or tubular member 20 that may define an atraumatic distal tip of guidewire 10. In at least some embodiments, tip member 28 may include a solder ball tip. Alternatively, tip member 28 may include a polymeric material. A coil (not shown) may be disposed in tubular member 20, for example adjacent tip member 28. In at least some embodiments, the coil may take the form of a radiopaque coil.

In at least some embodiments, tubular member 20 includes a plurality of cuts, apertures, and/or slots 42 formed therein. Various embodiments of arrangements and configurations of slots 42 are contemplated. In some embodiments, at least some, if not all of slots 42 are disposed at the same or a similar angle with respect to the longitudinal axis of the tubular member 20. As shown, slots 42 can be disposed at an angle that is perpendicular, or substantially perpendicular, and/or can be characterized as being disposed in a plane that is normal to the longitudinal axis of tubular member 20. However, in other embodiments, slots 42 can be disposed at an angle that is not perpendicular, and/or can be characterized as being disposed in a plane that is not normal to the longitudinal axis of tubular member 20. Additionally, a group of one or more slots 42 may be disposed at different angles relative to another group of one or more slots 42. The distribution and/or configuration of slots 42 can also include, to the extent applicable, any of those disclosed in U.S. Pat. Publication No. US 2004/0181174, the entire disclosure of which is herein incorporated by reference.

Slots 42 may be provided to enhance the flexibility of tubular member 20 while still allowing for suitable torque transmission characteristics. Slots 42 may be formed such that one or more rings and/or turns interconnected by one or more segments and/or beams are formed in tubular member 20, and such rings and beams may include portions of tubular member 20 that remain after slots 42 are formed in the body of tubular member 20. Such an interconnected ring structure may act to maintain a relatively high degree of tortional stiffness, while maintaining a desired level of lateral flexibility. In some embodiments, some adjacent slots 42 can be formed such that they include portions that overlap with each other about the circumference of tubular member 20. In other embodiments, some adjacent slots 42 can be disposed such that they do not necessarily overlap with each other, but are disposed in a pattern that provides the desired degree of lateral flexibility.

Additionally, slots 42 can be arranged along the length of, or about the circumference of, tubular member 20 to achieve desired properties. For example, adjacent slots 42, or groups of slots 42, can be arranged in a symmetrical pattern, such as being disposed essentially equally on opposite sides about the circumference of tubular member 20, or can be rotated by an angle relative to each other about the axis of tubular member 20. Additionally, adjacent slots 42, or groups of slots 42, may be equally spaced along the length of tubular member 20, or can be arranged in an increasing or decreasing density pattern, or can be arranged in a non-symmetric or irregular pattern. This may include slots 42 that form or otherwise follow a helical pattern about tubular member 20. Other characteristics, such as slot size, slot shape and/or slot angle with respect to the longitudinal axis of tubular member 20, can also be varied along the length of tubular member 20 in order to vary the flexibility or other properties. In other embodiments, moreover, it is contemplated that the portions of the tubular member, such as a proximal section 26, or a distal section 28, or the entire tubular member 20, may not include any such slots 42.

As suggested above, slots 42 may be formed in groups of two, three, four, five, or more slots 42, which may be located at substantially the same location along the axis of tubular member 20. Alternatively, a single slot 42 may be disposed at some or all of these locations. Within the groups of slots 42, there may be included slots 42 that are equal in size (i.e., span the same circumferential distance around tubular member 20). In some of these as well as other embodiments, at least some slots 42 in a group are unequal in size (i.e., span a different circumferential distance around tubular member 20). Longitudinally adjacent groups of slots 42 may have the same or different configurations. For example, some embodiments of tubular member 20 include slots 42 that are equal in size in a first group and then unequally sized in an adjacent group. It can be appreciated that in groups that have two slots 42 that are equal in size, the beams (i.e., the portion of tubular member 20 remaining after slots 42 are formed therein) are aligned with the center of tubular member 20. Conversely, in groups that have two slots 42 that are unequal in size, the beams are offset from the center of tubular member 20. Some embodiments of tubular member 20 include only slots 42 that are aligned with the center of tubular member 20, only slots 42 that are offset from the center of tubular member 20, or slots 42 that are aligned with the center of tubular member 20 in a first group and offset from the center of tubular member 20 in another group. The amount of offset may vary depending on the depth (or length) of slots 42 and can include essentially any suitable distance.

For the purposes of this disclosure, slots 42 may be understood to be cuts or openings that extend through the wall of tubular member 20. This, however, is not intended to be limiting as other arrangements are contemplated. For example, in some embodiments, pockets 42′ may be formed in tubular member 20′ instead of some or all of slots 42 as illustrated in FIG. 2A. For the purposes of this disclosure, pockets 42′ may be understood to be openings or cuts that are formed in tubular member 20′ that extend only part way through the wall of tubular member 20′. A pocket 42′ may alternatively be termed a trough, channel, groove, and the like, as appropriate. Pockets 42′ may have any of the configurations disclosed for slots 42 and/or any other suitable configuration.

Slots 42 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular member 20 is formed by cutting and/or removing portions of the tube to form slots 42. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guidewire 10 may include forming slots 42 in tubular member 20 using any of these or other manufacturing steps.

In still other embodiments, a hybrid laser/water jet cutting system may be used to form slots 42. An example of such a laser/water jet hybrid is commercially available from SYNOVA Inc., of Lausanne, Switzerland and is described in PCT Publication No. WO 9532834 entitled Device For Machining Material With A Laser and the corresponding U.S. Pat. No. 5,902,499, the entire disclosures of which are herein incorporated by reference.

In at least some embodiments, slots 42 may be formed in tubular member using a laser cutting process. The laser cutting process may include essentially any suitable laser and/or laser cutting apparatus. For example, the laser cutting process may utilize a fiber laser. Other lasers or laser systems may also be used, as appropriate. For example, if tubular member 20 is made from a polymer, it may be appropriate to forms slots 42 with an ultraviolet laser (e.g., with a shorter or “ultra fast” pulse time).

Utilizing processes like laser cutting may be desirable for a number of reasons. For example, laser cutting processes may allow tubular member 20 to be cut into a number of different cutting patterns in a precisely controlled manner. This may include variations in the slot width (which also may be termed “kerf”), ring width, beam height and/or width, etc. Furthermore, changes to the cutting pattern can be made without the need to replace the cutting instrument (e.g., a blade). This may also allow smaller tubes (e.g., having a smaller outer diameter) to be used to form tubular member 20 without being limited by a minimum cutting blade size. Consequently, tubular members 20 may be fabricated for use in neurological devices or other devices where a small size may be desired.

The laser cutting process may generally include providing tubular member 20 (i.e., the “uncut” or “unslotted” version of tubular member 20) and laser cutting a plurality of slots 42 in tubular member 20. These steps may include coupling or attaching tubular member 20 to a suitable holding apparatus. The holding apparatus may include one or more motors that can be used to rotate and/or translate tubular member 20 relative to the laser.

In addition to forming slots 42 in tubular member 20, tubular member 20 may also be subjected to additional method and/or processing steps. These steps may include a chemical treatment step. Chemical treatment may include any number of processes including chemical etching. Chemical etching may include, for example, bathing tubular member 20 in an acid bath. The acid bath may include essentially any suitable acid. For example, the acid bath may include fluoroboric acid, nitric acid, any suitable bench and/or mineral acid, combinations thereof, or any other suitable acid. In some embodiments, the acid bath may include an aqueous solution including fluoroboric acid (e.g., about 1-20% or more, or about 2-10% or more, or about 4% or more) and nitric acid (e.g., about 1-50% or more, or about 20-40% or more, or about 30% or more). It can be appreciated that the various solutions that may be appropriate for the various bathes may vary depending on the material(s) used for tubular member 20.

Chemical etching may be desirable for a number of reasons. For example, after the laser cutting process is completed, tubular member 20 may have waste products or “dross” disposed along the interior or exterior of tubular member 20. The dross may comprise the parts or scraps of tubular member 20 that are removed from tubular member 20 in forming slots 42. The dross may take the form of a dust-like or bead-like material that tends to accumulate along the various surfaces of tubular member 20. It may be desirable to remove dross as part of the manufacturing process.

In addition to removing dross, chemical etching may also remove portions of tubular member 20. This may be desirable for a number of reasons. For example, by virtue of removing portions of tubular member 20, chemical etching may be used to create a variety of different slot and/or ring configurations and/or orientations. This may include removing portions of tubular member 20 at or adjacent slots 42 via a chemical etching process. In addition, chemical etching may be used to vary the inner diameter, the outer diameter, or both of tubular member 20. This may include removing portions of tubular member 20 along the outer diameter, inner diameter, or both via a chemical etching process.

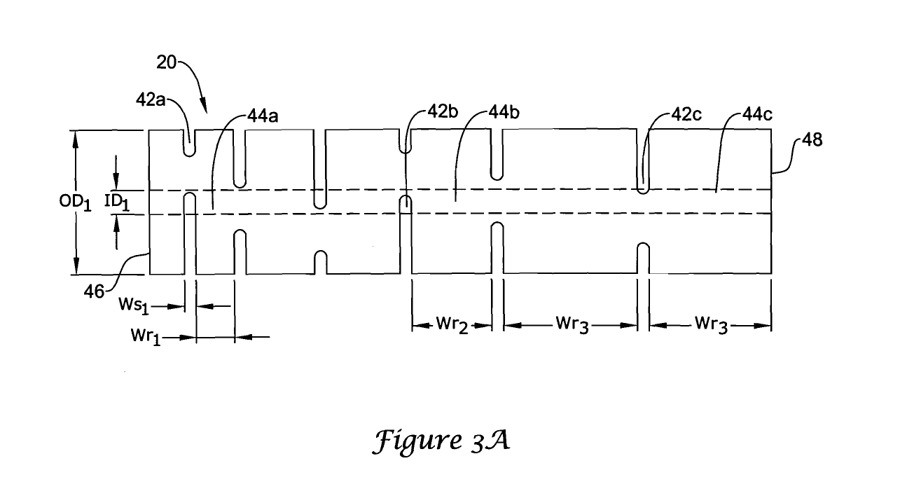

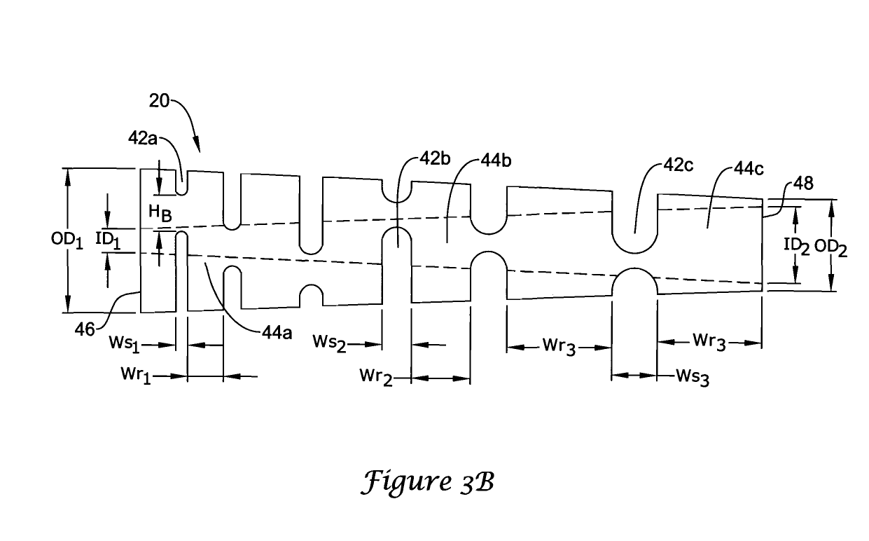

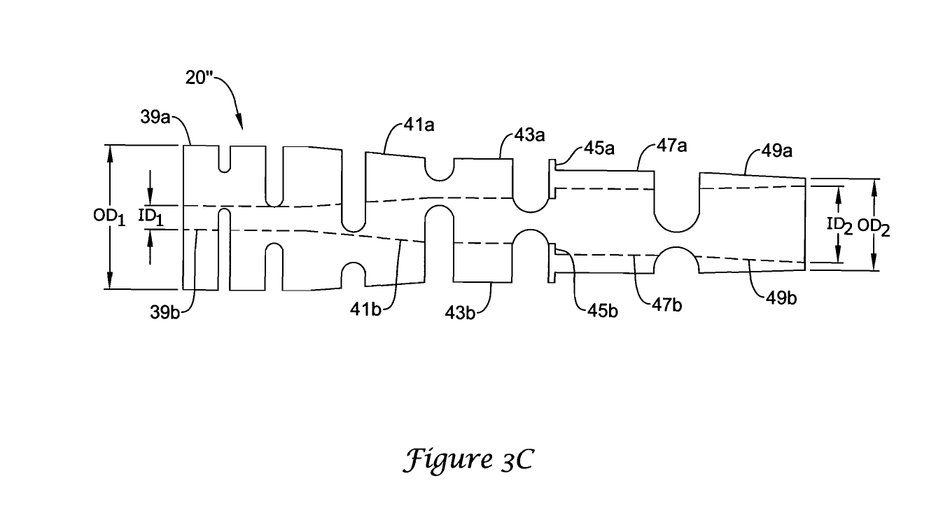

FIGS. 3A and 3B illustrate tubular member 20. In FIG. 3A, tubular member 20 is shown prior to a chemical etching step. Conversely, FIG. 3B illustrates tubular member 20 following a chemical etching step. At least some of the structural differences that may be incorporated into tubular member 20 using chemical etching can be seen by comparing FIG. 3A and FIG. 3B. The following description elaborates on some of the structural differences contemplated for tubular member 20 that may be incorporated into tubular member 20 using a chemical treatment and/or chemical etching step. It should be noted that these structural differences are provided for illustration purposes and are not intended to limit the scope of the invention. Any of the structural differences listed below may be used alone in example tubular members 20 or in combination with any of the other structural differences in other example tubular members 20. Furthermore, any one or more of these structural differences may be chosen for a particular tubular member 20 so as to provide the tubular member 20 with the desired level of torque-transmission, flexibility, trackability, and/or pushability.

In some embodiments, tubular member 20 may vary in its inner diameter. For example, tubular member 20 may have an first inner diameter ID₁ at one end. Prior to chemical etching, ID₁ may be constant along substantially the entire length of tubular member 20 as shown in FIG. 3A. As shown in FIG. 3B, tubular member 20 may also have a second inner diameter ID₂ at the opposite end. In some embodiments, ID₁ may be at the proximal end 46 of tubular member 20 and ID₂ may be disposed at the distal end 48 of tubular member 20. The reverse, of course, may also be true. The sizes of ID₁ and ID₂ may differ from one another after chemical etching. For example, ID₁ may be about 0.005 to about 0.015 inches (e.g., about 0.009 inches) and ID₂ may be about 0.005 to about 0.020 inches (e.g., about 0.010 inches). It can be appreciated that the inner diameter of tubular member 20 is shown to increase in the distal direction (i.e., approaching distal end 48). This, of course, can be reversed without departing from the spirit of the invention. The transition from ID₁ to ID₂ may occur gradually, constantly, variably, in a manner defined by a mathematical function, in stepped or step-wise manner, combinations thereof, or in any other suitable way.

Similarly, tubular member 20 may also have a variable outer diameter. For example, tubular member 20 may have an first outer diameter OD₁ at one end. Prior to chemical etching, OD₁ may be constant along substantially the entire length of tubular member 20 as shown in FIG. 3A. As shown in FIG. 3B, tubular member 20 may also have a second outer diameter OD₂ at the opposite end. In some embodiments, OD₁ may be at proximal end 46 of tubular member 20 and OD₂ may be disposed at distal end 48 of tubular member 20. The reverse, of course, may also be true. The sizes of OD₁ and OD₂ may differ from one another after chemical etching. For example, OD₁ may be about 0.010 to about 0.025 inches (e.g., about 0.014 inches) and OD₂ may be about 0.010 to about 0.020 inches (e.g., about 0.013 inches). It can be appreciated that the outer diameter of tubular member 20 is shown to decrease in the distal direction (i.e., approaching distal end 48). This, of course, can be reversed without departing from the spirit of the invention. The transition from OD₁ to OD₂ may occur gradually, constantly, variably, in a manner defined by a mathematical function, in stepped or step-wise manner, combinations thereof, or in any other suitable way.

The width or kerf of slots 42 can also vary. For example, FIGS. 3A and 3B illustrate slots 42a/42b/42c in tubular member 20. As shown in FIG. 3A, all of slots 42a/42b/42c may have substantially the same slot width Wₛ₁. After chemical etching, however, the slot widths may vary. For example, FIG. 3B illustrates that slot 42a has slot width Wₛ₁, slot 42b has a slot width Wₛ₂, and slot 42c has a slot width Wₛ₃. In some embodiments, the slot widths Wₛ₁/Wₛ₂/Wₛ₃ may increase in the distal direction (i.e., approaching distal end 48). In other embodiments, the slots widths Wₛ₁/Wₛ₂/Wₛ₃ may decrease in the distal direction. In still other embodiments, there may be both increases in decreases in the slot widths Wₛ₁/Wₛ₂/Wₛ₃ along the length of tubular member 20. Likewise the width of the rings (e.g., the portions tubular member 20 between longitudinally adjacent slots 42) can also vary. Alternatively, the slot widths may also be varied by varying the laser cutting process so that wider (or narrower) slots are formed, for example prior to chemical etching. Even though each of the various slots 42a/42b/42c illustrated in FIG. 3B has a constant width (e.g., they may have a generally square or rectangular shape), this is not intended to limit the invention. For example, embodiments are contemplated where the slot width is not constant or changes along the length or height of the slot. Such slots may have a tear drop shape, a “T-shape” (including an inverted “T-shape”), triangular shape, or the like.

Along with variations in slot width, ring width may also vary. In some embodiments, variation in ring width may occur by virtue of altering the spacing between longitudinally adjacent slots 42, for example, during the laser cutting process. In addition or in the alternative, variations in ring width may also be accomplished via chemical etching. For example, FIGS. 3A and 3B illustrates a number of rings including ring 44a having a ring width Wᵣ₁, ring 44b having a ring width Wᵣ₂, and ring 44c having a ring width Wᵣ₃. In some embodiments, the ring widths Wᵣ₁/Wᵣ₂/Wᵣ₃ may increase in the distal direction (i.e., approaching distal end 48). In other embodiments, the ring widths Wᵣ₁/Wᵣ₂/Wᵣ₃ may decrease in the distal direction. In still other embodiments, there may be both increases and decreases in the ring widths Wᵣ₁/Wᵣ₂/Wᵣ₃ along the length of tubular member 20. The transition from Wₛ₁ to Wₛ₂ to Wₛ₃ as well as the transition from Wᵣ₁ to Wᵣ₂ to Wᵣ₃ may occur gradually, constantly, variably, in a manner defined by a mathematical function, in stepped or step-wise manner, combinations thereof, or in any other suitable way.

In some embodiments, the beams (i.e., the portion of tubular member 20 disposed between a pair of slots 42 or the ends of a singular slot 42 at the same longitudinal position) may also vary. Variation in the beams may either be in the form of variable beam widths (which correspond to variations in slot width) and/or variation in beam heights. Although variations in beam height are not shown in FIGS. 3A and 3B merely for simplicity purposes, an example beam height H_{B} is noted in FIG. 3B so that this dimension can be readily identified. It can be appreciated that as the beam height increase, the slot “depths” (i.e., the depth to which the slots 42 go into tubular member 20) decrease. In some embodiments, all of the beams in tubular member 20 have the same height. Alternatively, at least some of the beams may have the different heights. In these later embodiments, the beams heights may change along the length of tubular member 20. For example, the beams heights may increase, decrease, or both along the length of tubular member 20.

FIG. 3B illustrates that the changes in the inner diameter and the outer diameter of tubular member 20 may occur gradually along the length of tubular member 20. This configuration, however, is not intended to be limiting. FIG. 3C illustrates tubular member 20″ that includes at least some of the contemplated alternative transitions in the inner and outer diameter. For example, tubular member 20″ may include a region 39a having a substantially constant outer diameter. Region 39a may transition to a region 41a having a linear or constant taper in outer diameter. Adjacent region 41a may be a region 43a having a constant outer diameter. Adjacent region 43a may be a region 45a having a stepwise change (e.g., including one or more steps) in outer diameter. Adjacent region 45a may be a region 47a having a constant outer diameter. Finally, adjacent region 47a may be a region 49a having a linear or constant taper in outer diameter. The arrangement of these regions 41a/43a/45a/47a/49a may differ from what is shown and multiple versions or copies of any particular region may be utilized in a given tubular member 20″.

The inner diameter of tubular member 20″ may also include a variety of different changes or transitions. For example, tubular member 20″ may include a region 39a having a substantially constant inner diameter. Region 39a may transition to a region 41a having a linear or constant taper in inner diameter. Adjacent region 41a may be a region 43a having a constant inner diameter. Adjacent region 43a may be a region 45a having a stepwise change (e.g., including one or more steps) in inner diameter. Adjacent region 45a may be a region 47a having a constant inner diameter. Finally, adjacent region 47a may be a region 49a having a linear or constant taper in inner diameter. Just like what is stated above in relation to the changes in outer diameter, the arrangement of regions 41b/43b/45b/47b/49b may differ from what is shown and multiple versions or copies of any particular region may be utilized in a given tubular member 20″.

It can be appreciated that any of the various transitions and/or changes in the outer and inner diameter of tubular member 20″ may be utilized in any of the tubular members disclosed herein. Creating such transitions may include the use of chemical etching and/or mass removal as disclosed herein. To achieve the various alterations, a variety of masking agents or devices may be used that the rate at which mass is removed can be altered to achieve the desired result. For example, a mask may be applied to region 39a/39b, which may prevent mass removal at these locations and allow for them to have a constant outer diameter. It can be appreciated that numerous designs can be created or “customized” for the particular needs of a given intervention.

It may be desirable to perform one or more cleaning steps on tubular member 20 in addition to the laser cutting process and chemical etching process. These cleaning steps may degrease, rinse, de-dross, and/or otherwise clean tubular member 20. Some of these steps may include, for example, bathing tubular member 20 in an alkaline bath (e.g., degreasing tubular member 20 in an alkaline bath), bathing tubular member 20 in an acid bath (e.g., de-drossing tubular member 20 in an acid bath), bathing and/or cleaning tubular member 20 (e.g., bathing, bathing in water, sonication cleaning, water jet spray bathing, etc.), drying tubular member 20 (e.g., forced air drying, etc.), and/or the like, and/or any other suitable step. In some embodiments, the cleaning steps may include degreasing tubular member 20 with an alkaline bath, de-drossing tubular member 20 in an acid bath, sonically cleaning tubular member 20, bathing tubular member 20 with water, and drying tubular member 20.

The chemicals used for the various baths may vary. For example, the alkaline bath may include an alkali and/or alkaline earth metal hydroxide solution that may titrated to the desired pH. Essentially any other suitable alkaline bath may be used without departing from the spirit of the invention. In some embodiments, the alkaline bath may be buffered using a suitable buffer solution. The acid bath may include essentially any suitable acid and/or baths including those listed above. It can be appreciated that the various solutions that may be appropriate for the various bathes may vary depending on the material(s) used for tubular member 20.

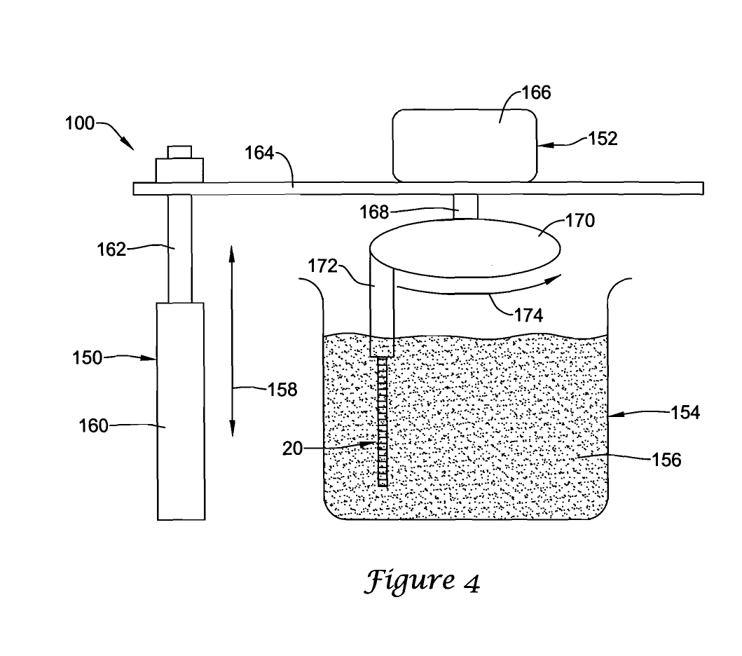

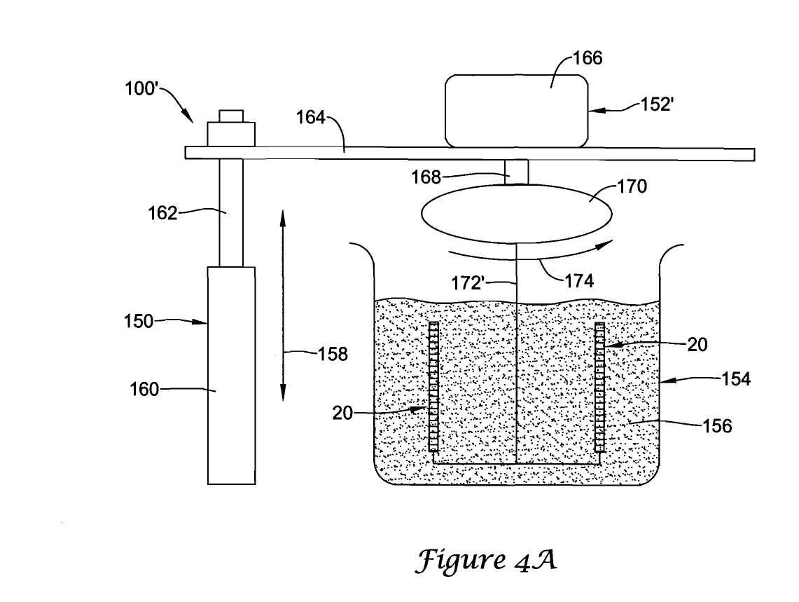

In at least some embodiments, the chemical etching step (as well as one or more of the bathing steps) may occur while moving tubular member 20 in and out of the solution and/or rotating tubular member 20. This may be accomplished using a suitable apparatus 100, which is illustrated in FIG. 4. Apparatus 100 may include a first actuation member 150 and a second actuation member 152. A bath 154 containing the appropriate bath material 156 (e.g., an acid bath suitable for chemical etching) may be disposed adjacent members 150/152. First actuation member 150 may be configured to move tubular member 20 up and down (e.g., as indicated by arrow 158) so that tubular member 20 may move into and out from bath material 156. Because tubular member 20 may be oriented in an upright or vertical position, this movement may also be understood to be translating tubular member 20 or translating tubular member 20 into and out from bath material 156. In some embodiments, first actuation member 150 may include a pneumatic cylinder 160 having a vertical arm 162 and a horizontal arm 164. Vertical arm 162 may extend from and be configured to move up and down within cylinder 160. Horizontal arm 164 may extend to second actuation member 152, which ultimately connects to tubular member 20. In some of these as well as in some other embodiments, first actuation member 150 may include a precision motion control motor or means. However, essentially any suitable machine for providing translation (e.g., linear translation) may be utilized including a linear motor, a ball screw such as a motor driven ball screw, an acme screw, and the like, or combinations thereof.

Second actuation member 152 may be configured to rotate tubular member 20 (e.g., as indicated by arrow 174) within bath material 156. In some embodiments, second actuation member 152 may include a rotary motor 166, a vertical arm 168, a rotary base 170, and a chuck or mounting member 172. Rotary motor 166 may be disposed on or adjacent arm 164. Vertical arm 168 may extend through or otherwise below arm 164 and attach to base 170. Chuck 172 may attach to base 170. In some embodiments, chuck 172 is configured to secure and/or hold tubular member 20. This may include holding tubular member 20 via a locking chuck, a clamp, a grip, a fastener, or any other suitable structure. For example, in some alternative embodiments of apparatus 100′, second actuation member 152′ may include a rack 172′ that holds one or more tubular members 20 as shown in FIG. 4A. Portions of rack 172′ may extend into the interior of tubular members 20, which may help hold tubular member(s) in place. Numerous other apparatuses are contemplated that may use any number of a variety of differing second actuation members.

In use, apparatus 100 (and/or other apparatuses disclosed herein) may be set up with tubular member 20 coupled to chuck 172 and with the suitable bath material 156 (e.g., an acid bath suitable for chemical etching) in bath 154. When properly set up, tubular member 20 may be etched by bringing tubular member 20 into and out from bath material 156 (e.g., by moving arm 162 up and down), rotating tubular member 20 (e.g., by rotating base 170), or both. In some embodiments, the speeds that members 150/152 translate and/or rotate tubular member 20 may vary. In general, the rate of motion may correlate to the rate of mass-removal from tubular member 20. For example, the translation rate may vary in steps of 1-4 inches/minute or may be at continuously varying speeds of 0.5-2.5 inches/minute. As an example, the following translation rates may result in the corresponding percent of mass lost via the etching process: 1 inch/minute, 25.9%; 0.8 inches/minute, 28.3%; 0.66 inches/minute, 33%; and 0.57 inches/minute, 36.7%. The rotation rate may be about 2-20 rpm or greater (e.g., about 5 rpm or greater). These rates may be appropriate for a variety of tubular member 20 lengths including as small as 1 inches long or smaller. It can be appreciated that any of these rates may vary without departing from the spirit of the invention. Moreover, in some embodiments an electrochemical process (e.g., that may add current to bath 154) may be utilized. These processes may alter (e.g., increase) the rate of mass removal.

It can be appreciated that the longer that tubular member 20 is in bath material 156, the greater amount of material that may be removed from tubular member 20. Thus, if one end of tubular member 20 spends more time in bath material, more material along that end may be removed (resulting in greater changes in inner diameter, outer diameter, slot width, or combinations thereof) that at the other end. This property may be altered, for example, by masking portions of tubular member 20 so that one or more structural characteristic (e.g., inner diameter, outer diameter, slot width, etc.) may be left unaltered be altered to a lesser extent.

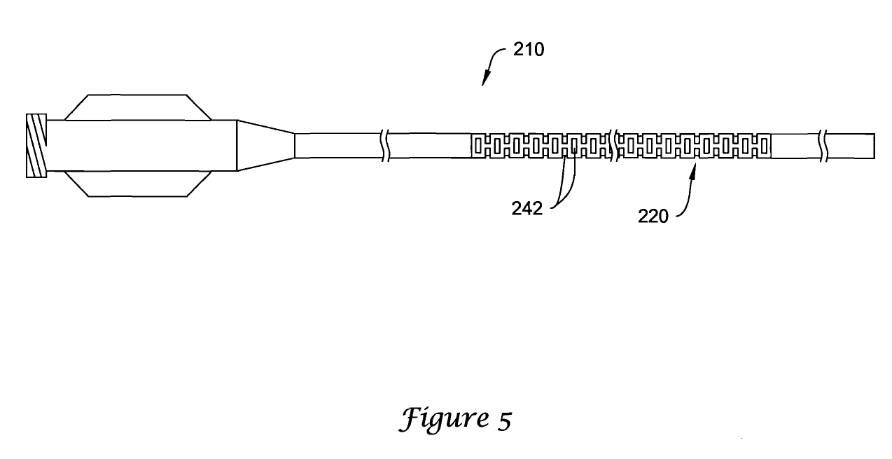

The forgoing discussion indicates that in at least some embodiments, tubular member 20 may be a part of guidewire 10. However, this is not intended to limit the scope of the invention as tubular member 20 may be used in essentially any other suitable medical device. For example, FIG. 5 illustrates another example medical device 210 that takes the form of a catheter and may include a tubular member 220, which may be similar in form and function to tubular member 20. For example, tubular member 220 may include a plurality of slots 242, which may be configured and/or arranged similarly to slots 42.

The materials that can be used for the various components of guidewire 10 may include those commonly associated with medical devices. For example, core wire 18, and/or tubular member 20, and/or connector 26, and/or tip member 28, and the like may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; niobium, niobium alloys, combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANTUM™ (available from Neo-Metrics) and GUM METAL™ (available from Toyota). In some other embodiments, a superelastic alloy, for example a superelastic nitinol can be used to achieve desired properties.

In at least some embodiments, portions or all of core wire 18 and/or tubular member 20 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of MRI compatibility is imparted into guidewire 10. For example, to enhance compatibility with Magnetic Resonance Imaging (MRI) machines, it may be desirable to make core wire 18 and/or tubular member 20, or other portions of the guidewire 10, in a manner that would impart a degree of MRI compatibility. For example, core wire 18 and/or tubular member 20, or portions thereof, may be made of a material that does not substantially distort the image and create substantial artifacts (artifacts are gaps in the image). Certain ferromagnetic materials, for example, may not be suitable because they may create artifacts in an MRI image. Core wire 18 and/or tubular member 20, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Referring now to core wire 18, the entire core wire 18 can be made of the same material along its length, or in some embodiments, can include portions or sections made of different materials. In some embodiments, the material used to construct core wire 18 is chosen to impart varying flexibility and stiffness characteristics to different portions of core wire 18. For example, proximal section 22 and distal section 24 of core wire 18 may be formed of different materials, for example materials having different moduli of elasticity, resulting in a difference in flexibility. In some embodiments, the material used to construct proximal section 22 can be relatively stiff for pushability and torqueability, and the material used to construct distal section 24 can be relatively flexible by comparison for better lateral trackability and steerability. For example, proximal section 22 can be formed of straightened 304v stainless steel wire or ribbon and distal section 24 can be formed of a straightened super elastic or linear elastic alloy, for example a nickel-titanium alloy wire or ribbon.

In embodiments where different portions of core wire 18 are made of different materials, the different portions can be connected using any suitable connecting techniques and/or with connector 26. For example, the different portions of core wire 18 can be connected using welding (including laser welding), soldering, brazing, adhesive, or the like, or combinations thereof. These techniques can be utilized regardless of whether or not connector 26 is utilized. Connector 26 may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Essentially any suitable configuration and/or structure can be utilized for connector 26 including those connectors described in U.S. Pat. Nos. 6,918,882 and 7,071,197 and/or in U.S. Patent Pub. No. 2006-0122537, the entire disclosures of which are herein incorporated by reference.

A sheath or covering (not shown) may be disposed over portions or all of core wire 18 and/or tubular member 20 that may define a generally smooth outer surface for guidewire 10. In other embodiments, however, such a sheath or covering may be absent from a portion of all of guidewire 10, such that tubular member 20 and/or core wire 18 may form the outer surface. The sheath may be made from a polymer or any other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the guidewire 10 (including, for example, the exterior surface of core wire 18 and/or the exterior surface of tubular member 20) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of core wire 18 and/or tubular member, or other portions of device 10. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The same may be true of tip member 30. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

In some embodiments, an oxide coating may be applied to the exterior surface of the guidewire 10 (including, for example, the exterior surface of core wire 18 and/or the exterior surface of tubular member 20). For example, a titanium oxide, iridium oxide, or other coating may be applied, for example, using any suitable technique such as plasma vapor deposition, chemical vapor deposition, or the like. An oxide coating may increase the durability and/or wear resistance of guidewire 10 and/or the components thereof.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method for manufacturing a medical device, the method comprising:
providing a metallic tubular member, wherein the tubular member includes an outer diameter and an inner diameter;
cutting a plurality of slots in the tubular member, wherein the slots have a slot width;
chemically treating at least a portion of the tubular member to remove material from the tubular member and define a treated portion; and
wherein the treated portion has one or more of a decreased outer diameter, an increased inner diameter, or an increased slot width in at least some of the slots.

2. The method of claim 1, wherein the tubular member includes a nickel-titanium alloy.

3. The method of claim 1, wherein cutting a plurality of slots in the tubular member includes laser cutting.

4. The method of claim 3, wherein laser cutting includes laser cutting with a fiber laser.

5. The method of claim 1, wherein chemically treating at least a portion of the tubular member to remove material from the tubular member and define a treated portion includes bathing the tubular member in fluoroboric acid.

6. The method of claim 1, wherein chemically treating at least a portion of the tubular member to remove material from the tubular member and define a treated portion includes bathing the tubular member in nitric acid.

7. The method of claim 1, wherein chemically treating at least a portion of the tubular member to remove material from the tubular member and define a treated portion includes bathing the tubular member in an acid bath while rotating the tubular member, translating the tubular member, or both.

8. The method of claim 1, wherein chemically treating at least a portion of the tubular member to remove material from the tubular member and define a treated portion includes moving the tubular member in and out of an acid bath.

9. The method of claim 1, further comprising sonically bathing the tubular member.

10. The method of claim 1, further comprising bathing the tubular member with water.

11. The method of claim 10, wherein bathing the tubular member with water includes bathing with a water jet spray.

12. The method of claim 1, further comprising forced air drying the tubular member.

13. The method of claim 1, further comprising degreasing the tubular member with an alkaline bath.

14. The method of claim 1, further comprising de-drossing the tubular member in an acid bath.

15. A method for manufacturing a medical device, the method comprising:
providing a nickel-titanium alloy tubular member, the tubular member having an outer diameter and an inner diameter;
laser cutting a plurality of slots in the tubular member, wherein the slots have a slot width; and
chemically etching at least a portion of the tubular member to remove material from the tubular member and define a treated portion, the treated portion having a decreased outer diameter, an increased inner diameter, and an increased slot width in at least some of the slots.

16. The method of claim 15, wherein laser cutting a plurality of slots in the tubular member includes laser cutting with a fiber laser.

17. The method of claim 15, wherein chemically etching at least a portion of the tubular member to remove material from the tubular member and define a treated portion includes bathing the tubular member in fluoroboric acid.

18. The method of claim 15, wherein chemically etching at least a portion of the tubular member to remove material from the tubular member and define a treated portion includes bathing the tubular member in nitric acid.

19. The method of claim 15, wherein chemically etching at least a portion of the tubular member to remove material from the tubular member and define a treated portion includes bathing the tubular member in an acid bath while rotating the tubular member, translating the tubular member, or both.

20. The method of claim 15, wherein chemically etching at least a portion of the tubular member to remove material from the tubular member and define a treated portion includes moving the tubular member in and out of an acid bath.

21. A medical device, comprising:
an elongate shaft including a metallic tubular member, the tubular member having a length, an inner diameter, an outer diameter, and a plurality of slots formed therein; and
wherein the inner diameter varies along the length of the tubular member.

22. The medical device of claim 21, wherein the outer diameter varies along the length of the tubular member.

23. The medical device of claim 21, wherein the slots have a slot width and wherein the slot width varies along the length of the tubular member.

24. The medical device of claim 21, wherein a first ring is defined in the tubular member between a first set of longitudinally adjacent slots, the first ring having a first length, wherein a second ring is defined in the tubular member between a second set of longitudinally adjacent slots, wherein the first ring is longitudinally spaced from the second ring, wherein the first ring has a first length, and wherein the second ring has a second length different from the first length.

25. The medical device of claim 21, wherein the tubular member has a proximal end and a distal end, and wherein the inner diameter is larger adjacent the proximal end than adjacent the distal end.

26. The medical device of claim 21, wherein the tubular member has a proximal end and a distal end, and wherein the inner diameter is larger adjacent the distal end than adjacent the proximal end.

27. The medical device of claim 21, wherein the tubular member has a proximal end and a distal end, and wherein the outer diameter is larger adjacent the distal end than adjacent the proximal end.

28. The medical device of claim 21, wherein the tubular member has a proximal end and a distal end, and wherein the outer diameter is larger adjacent the proximal end than adjacent the distal end.

29. A method for manufacturing a medical device, the method comprising:
providing a tubular member, wherein the tubular member includes an outer diameter and an inner diameter;
cutting a plurality of slots in the tubular member, wherein the slots have a slot width;
chemically treating at least a portion of the tubular member to remove material from the tubular member and define a treated portion; and
wherein the treated portion has one or more of a decreased outer diameter, an increased inner diameter, or an increased slot width in at least some of the slots.

30. A medical device, comprising:
an elongate shaft including a tubular member, the tubular member having a length, an inner diameter, an outer diameter, and a plurality of slots formed therein;
wherein the inner diameter varies along the length of the tubular member; and
wherein the outer diameter varies along the length of the tubular member.

31. A method for manufacturing a medical device, the method comprising:
providing a tubular member, wherein the tubular member includes an outer diameter and an inner diameter;
forming a plurality of pockets in the tubular member, wherein the pockets have a pocket width;
chemically treating at least a portion of the tubular member to remove material from the tubular member and define a treated portion; and
wherein the treated portion has one or more of a decreased outer diameter, an increased inner diameter, or an increased pocket width in at least some of the pockets.
